SEQUENCE LISTING (12 sequences)

SEQ ID NO: 1 - DNA, 2813 bp, Mus musculus
  CDS (40)..(2469)
gcgttggtgcgccctgcttggcggggggcctccggagcgatgccgatggatgtgattttagttttgtggttctgtgtgtgcaccgccaggacagtgctgggctttgggatggaccctgaccttcagatggacatcatcactgaacttgaccttgtgaacaccaccctgggcgtcactcaggtggctggactacacaatgccagtaaggcatttctgtttcaagatgtacagagagagatccactcagcccctcatgtgagtgagaagctgatccagctattccggaataagagtgagtttacctttttggctacagtgcagcagaagccgtccacctcaggggtgatactgtcgatccgggagctggaacacagctattttgaactggagagcagtggcccaagagaagagatacgctatcattacatccatggcggcaagcccaggactgaggcccttccctaccgcatggccgatggacagtggcacaaggtcgcgctgtctgtgagcgcctctcacctcctactccatgtcgactgcaataggatttatgagcgtgtgatagatcctccggagaccaaccttcctccaggaagcaatctatggcttgggcaacgtaatcaaaagcatggctttttcaaaggaatcatccaagatggcaagatcatcttcatgccgaacggcttcatcacacagtgccccaacctaaatcgcacttgcccaacatgcagtgatttcctgagcctggttcaaggaataatggatttgcaagagcttttggccaagatgactgcaaaactgaattatgcagagacgagacttggtcaactggaaaattgccactgtgagaagacctgccaagtgagtgggctgctctacagggaccaagactcctgggtagatggtgacaactgcaggaactgcacatgcaaaagtggtgctgtggagtgccgaaggatgtcctgtcccccactcaactgttccccagactcacttcctgtgcatatttctggccaatgttgtaaagtttgcagaccaaaatgtatctatggaggaaaagttcttgctgagggccagcggattttaaccaagacctgccgggaatgtcgaggtggagtcttggtaaaaatcacagaagcttgccctcctttgaactgctcagagaaggatcatattcttccggagaaccagtgctgcagggtctgccgaggtcataacttctgtgcagaagcacctaagtgtggagaaaactcggaatgcaaaaattggaatacaaaagcgacttgtgagtgcaagaatggatacatctctgtccagggcaactctgcatactgtgaagatatcgatgagtgtgcagcaaagatgcactactgtcatgccaacacggtgtgtgtcaacttgccggggttatatcgctgtgactgcatcccaggatacatccgtgtggatgacttctcttgtacggagcatgatgattgtggcagcggacaacacaactgtgacaaaaatgccatctgtaccaacacagtccagggacacagctgtacctgccagccaggctacgtgggaaatggtactgtctgcaaagcattctgtgaagagggttgcagatacggaggtacctgtgtggcccctaacaaatgtgtctgtccttctggattcacaggaagccactgtgagaaagatattgatgaatgtgcagagggattcgttgagtgccacaaccactcccgctgcgttaaccttccagggtggtaccactgtgagtgcagaagcggtttccatgacgatgggacctattcactgtccggggagtcctgcattgatattgatgaatgtgccttaagaactcacacttgttggaatgactctgcctgcatcaacttagcaggaggatttgactgcctgtgtccctctgggccctcctgctctggtgactgtccccacgaaggggggctgaagcataatgggcaggtgtggattctgagagaagacaggtgttcagtctgttcctgtaaggatgggaagatattctgccggcggacagcttgtgattgccagaatccaaatgttgaccttttctgctgcccagagtgtgacaccagggtcactagccaatgtttagatcaaagcggacagaagctctatcgaagtggagacaactggacccacagctgccagcagtgccgatgtctggaaggagaggcagactgctggcctctagcttgccctagtttgagctgtgaatacacagccatctttgaaggagagtgttgtccccgctgtgtcagtgacccctgcctggctgataatattgcctatgacatcagaaaaacttgcctggacagctctggtatttcgaggctgagcggcgcagtgtggacaatggctggatctccctgtacaacctgtcaatgcaagaatgggagagtctgctgctctgtggatctggtgtgtcttgagaataactgaagattttaaatggactcatcacatgagaaaatggacaaaatgaccatctaacctgaggaagaggaggggctgatttctttttctttttaaccacagtcaattaccaaagtctccatcagaggaaggcgtttgggttgcctttaccactttgctcatccttgctgacctagtctagatgcctgcagtaccgtgtatttcggtcgatggttgttgagtctccgtgctgtaaatcacattccccttgtcagatcatttacagatacatttaaaggattccatgataaatgttaaagtaccttttgtttattttgtgtactgacataatagagacttggcaccaa

SEQ ID NO: 2 - protein, 810 aa, Mus musculus
MPMDVILVLWFCVCTARTVLGFGMDPDLQMDIITELDLVNTTLGVTQVAGLHNASKAFLFQDVQREIHSAPHVSEKLIQLFRNKSEFTFLATVQQKPSTSGVILSIRELEHSYFELESSGPREEIRYHYIHGGKPRTEALPYRMADGQWHKVALSVSASHLLLHVDCNRIYERVIDPPETNLPPGSNLWLGQRNQKHGFFKGIIQDGKIIFMPNGFITQCPNLNRTCPTCSDFLSLVQGIMDLQELLAKMTAKLNYAETRLGQLENCHCEKTCQVSGLLYRDQDSWVDGDNCRNCTCKSGAVECRRMSCPPLNCSPDSLPVHISGQCCKVCRPKCIYGGKVLAEGQRILTKTCRECRGGVLVKITEACPPLNCSEKDHILPENQCCRVCRGHNFCAEAPKCGENSECKNWNTKATCECKNGYISVQGNSAYCEDIDECAAKMHYCHANTVCVNLPGLYRCDCIPGYIRVDDFSCTEHDDCGSGQHNCDKNAICTNTVQGHSCTCQPGYVGNGTVCKAFCEEGCRYGGTCVAPNKCVCPSGFTGSHCEKDIDECAEGFVECHNHSRCVNLPGWYHCECRSGFHDDGTYSLSGESCIDIDECALRTHTCWNDSACINLAGGFDCLCPSGPSCSGDCPHEGGLKHNGQVWILREDRCSVCSCKDGKIFCRRTACDCQNPNVDLFCCPECDTRVTSQCLDQSGQKLYRSGDNWTHSCQQCRCLEGEADCWPLACPSLSCEYTAIFEGECCPRCVSDPCLADNIAYDIRKTCLDSSGISRLSGAVWTMAGSPCTTCQCKNGRVCCSVDLVCLENN

SEQ ID NO: 3 - DNA, 2541 bp, Homo sapiens
  CDS (64)..(2493)
tccaggctcatttgcttccacctagcttcggtgccccctgctaggcggggaccctcgagagcgatgccgatggatttgattttagttgtgtggttctgtgtgtgcactgccaggacagtggtgggctttgggatggaccctgaccttcagatggatatcgtcaccgagcttgaccttgtgaacaccacccttggagttgctcaggtgtctggaatgcacaatgccagcaaagcatttttatttcaagacatagaaagagagatccatgcagctcctcatgtgagtgagaaattaattcagctgttccggaacaagagtgaattcaccattttggccactgtacagcagaagccatctacttcaggagtgatactgtccattcgagaactggagcacagctattttgaactggagagcagtggcctgagggatgagattcggtatcactacatacacaatgggaagccaaggacagaggcacttccttaccgcatggcagatggacaatggcacaaggttgcactgtcagttagcgcctctcatctcctgctccatgtcgactgtaacaggatttatgagcgtgtgatagaccctccagataccaaccttcccccaggaatcaatttatggcttggccagcgcaaccaaaagcatggcttattcaaagggatcatccaagatgggaagatcatctttatgccgaatggatatataacacagtgtccaaatctaaatcacacttgcccaacctgcagtgatttcttaagcctggtgcaaggaataatggatttacaagagcttttggccaagatgactgcaaaactaaattatgcagagacaagacttagtcaattggaaaactgtcattgtgagaagacttgtcaagtgagtggactgctctatcgagatcaagactcttgggtagatggtgaccattgcaggaactgcacttgcaaaagtggtgccgtggaatgccgaaggatgtcctgtccccctctcaattgctccccagactccctcccagtgcacattgctggccagtgctgtaaggtctgccgaccaaaatgtatctatggaggaaaagttcttgcagaaggccagcggattttaaccaagagctgtcgggaatgccgaggtggagttttagtaaaaattacagaaatgtgtcctcctttgaactgctcagaaaaggatcacattcttcctgagaatcagtgctgccgtgtctgtagaggtcataacttttgtgcagaaggacctaaatgtggtgaaaactcagagtgcaaaaactggaatacaaaagctacttgtgagtgcaagagtggttacatctctgtccagggagactctgcctactgtgaagatattgatgagtgtgcagctaagatgcattactgtcatgccaatactgtgtgtgtcaaccttcctgggttatatcgctgtgactgtgtcccaggatacattcgtgtggatgacttctcttgtacagaacacgatgaatgtggcagcggccagcacaactgtgatgagaatgccatctgcaccaacactgtccagggacacagctgcacctgcaaaccgggctacgtggggaacgggaccatctgcagagctttctgtgaagagggctgcagatacggtggaacgtgtgtggctcccaacaaatgtgtctgtccatctggattcacaggaagccactgcgagaaagatattgatgaatgttcagagggaatcattgagtgccacaaccattcccgctgcgttaacctgccagggtggtaccactgtgagtgcagaagcggtttccatgacgatgggacctattcactgtccggggagtcctgtattgacattgatgaatgtgccttaagaactcacacctgttggaacgattctgcctgcatcaacctggcagggggttttgactgtctctgcccctctgggccctcctgctctggtgactgtcctcatgaaggggggctgaagcacaatggccaggtgtggaccttgaaagaagacaggtgttctgtctgctcctgcaaggatggcaagatattctgccgacggacagcttgtgattgccagaatccaagtgctgacctattctgttgcccagaatgtgacaccagagtcacaagtcaatgtttagaccaaaatggtcacaagctgtatcgaagtggagacaattggacccatagctgtcagcagtgtcggtgtctggaaggagaggtagattgctggccactcacttgccccaacttgagctgtgagtatacagctatcttagaaggggaatgttgtccccgctgtgtcagtgacccctgcctagctgataacatcacctatgacatcagaaaaacttgcctggacagctatggtgtttcacggcttagtggctcagtgtggacgatggctggatctccctgcacaacctgtaaatgcaagaatggaagagtctgttgttctgtggattttgagtgtcttcaaaataattgaagtatttacagtggactcaacgcagaagaatggacgaaatgacca

SEQ ID NO: 4 - protein, 810 aa, Homo sapiens
MPMDLILVVWFCVCTARTVVGFGMDPDLQMDIVTELDLVNTTLGVAQVSGMHNASKAFLFQDIEREIHAAPHVSEKLIQLFRNKSEFTILATVQQKPSTSGVILSIRELEHSYFELESSGLRDEIRYHYIHNGKPRTEALPYRMADGQWHKVALSVSASHLLLHVDCNRIYERVIDPPDTNLPPGINLWLGQRNQKHGLFKGIIQDGKIIFMPNGYITQCPNLNHTCPTCSDFLSLVQGIMDLQELLAKMTAKLNYAETRLSQLENCHCEKTCQVSGLLYRDQDSWVDGDHCRNCTCKSGAVECRRMSCPPLNCSPDSLPVHIAGQCCKVCRPKCIYGGKVLAEGQRILTKSCRECRGGVLVKITEMCPPLNCSEKDHILPENQCCRVCRGHNFCAEGPKCGENSECKNWNTKATCECKSGYISVQGDSAYCEDIDECAAKMHYCHANTVCVNLPGLYRCDCVPGYIRVDDFSCTEHDECGSGQHNCDENAICTNTVQGHSCTCKPGYVGNGTICRAFCEEGCRYGGTCVAPNKCVCPSGFTGSHCEKDIDECSEGIIECHNHSRCVNLPGWYHCECRSGFHDDGTYSLSGESCIDIDECALRTHTCWNDSACINLAGGFDCLCPSGPSCSGDCPHEGGLKHNGQVWTLKEDRCSVCSCKDGKIFCRRTACDCQNPSADLFCCPECDTRVTSQCLDQNGHKLYRSGDNWTHSCQQCRCLEGEVDCWPLTCPNLSCEYTAILEGECCPRCVSDPCLADNITYDIRKTCLDSYGVSRLSGSVWTMAGSPCTTCKCKNGRVCCSVDFECLQNN

SEQ ID NO: 5 - DNA, 2915 bp, Rattus norvegicus
  CDS (59)..(2488)
aagcactggtttcttgttagcgttggtgcgccctgcttggcgggggttctccggagcgatgccgatggatgtgattttagttttgtggttctgtgtatgcaccgccaggacagtgttgggctttgggatggaccctgaccttcagctggacatcatctcagagctcgacctggtgaacaccaccctgggagtcacgcaggtggctggactgcacaacgccagtaaagcatttctatttcaagatgtacagagagagatccattcggcccctcacgtgagtgagaagctgatccagctattccggaataagagcgagttcacctttttggctacagtgcagcagaaaccatccacctcaggggtgatactgtccatccgggagctggagcacagctattttgaactggagagcagtggcccaagagaagagatacgctaccattacatacatggtggaaagcccaggactgaggcccttccctaccgcatggcagacggacaatggcacaaggtcgcgctgtcagtgagcgcctctcacctcctgctccacatcgactgcaataggatttacgagcgtgtgatagaccctccggagaccaaccttcctccaggaagcaatctgtggcttgggcaacgtaaccaaaagcatggctttttcaaaggaatcatccaagatggtaagatcatcttcatgccgaatggtttcatcacacagtgtcccaacctcaatcgcacttgcccaacatgcagtgacttcctgagcctggttcaaggaataatggatttgcaagagcttttggccaagatgactgcaaaactgaattatgcagagacgagacttggtcaactggaaaattgccactgtgagaagacctgccaagtgagtgggctgctctacagggaccaagactcctgggtggatggtgacaactgtgggaactgcacgtgcaaaagtggtgccgtggagtgccgcaggatgtcctgtcccccgctcaactgttccccggactcacttcctgtgcacatttccggccagtgttgtaaagtttgcagaccaaaatgtatctatggaggaaaagttcttgctgagggccagcggattttaaccaagacctgccgggaatgtcgaggtggagtcttggtaaaaatcacagaagcttgccctcctttgaactgctcagcaaaggatcatattcttccagagaatcagtgctgcagggtctgcccaggtcataacttctgtgcagaagcacctaagtgcggagaaaactcggaatgcaaaaattggaatacaaaagcaacctgtgagtgcaagaatggatacatctctgtccagggcaactctgcatactgtgaagatattgatgagtgtgcagctaaaatgcactattgtcatgccaacaccgtgtgtgtcaacttgccggggttgtatcgctgtgactgcgtcccagggtacatccgtgtggatgacttctcttgtacggagcatgatgattgtggcagcggacaacacaactgcgacaaaaatgccatctgtaccaacacagtccagggacacagctgcacctgccagccgggttacgtgggaaatggcaccatctgcaaagcattctgtgaagagggttgcagatacggaggtacctgtgtggctcctaacaagtgtgtctgtccttctggattcacgggaagccactgtgagaaagatattgatgaatgcgcagagggattcgttgaatgccacaactactcccgctgtgttaacctgccagggtggtaccactgtgagtgcagaagcggtttccatgacgatgggacctactcactgtccggggagtcctgcattgatatcgatgaatgtgccttaagaactcacacttgttggaatgactctgcctgcatcaacttagcaggaggatttgactgcctgtgtccctctgggccctcctgctctggtgactgtccccacgaaggagggctgaagcataatgggcaggtgtggattctgagagaagacaggtgttcagtctgttcctgcaaggatgggaagatattctgccggcggacagcttgtgattgccagaatccaaatgttgaccttttttgctgcccagagtgcgataccagggtcaccagccaatgtttagatcaaagtggacagaagctctatcgaagtggagacaactggacccacagctgccagcagtgccgatgtctggaaggagaggcagactgctggcctctggcttgccctagtttgggctgtgaatacacagccatgtttgaaggggagtgttgtccccgatgtgtcagtgacccctgcctggctggtaatattgcctatgacatcagaaaaacttgcctggacagctttggtgtttcgaggctgagcggagccgtgtggacaatggctggatctccttgtacaacctgcaaatgcaagaatgggagagtctgctgctctgtggatctggagtgtattgagaataactgaagattttaaatggactcgtcacgtgagaaaatgggcaaaatgatcatcccacctgaggaagaagaggggctgatttctttttctttttaaccacagtcaattaccaaagtctccatctgaggaaggcgtttggattgcctttgccactttgctcatccttgctgacctagtctagatgcctgcagtaccgtgcatttcggtcgatggttgttgagtctcagtgttgtaaatcgcatttccctcgtcagatcatttacagatacatttaaaggggttccatgataaatgttaatgtaacttttgtttattttgtgtactgacataatagagacttggcaccatttatttatttttcttgatttttggatcaaattctaaaaataaagttgcctgttgcgaaaaaaaaaaaaaaaaaaaaaaaaaa

SEQ ID NO: 6 - protein, 810 aa, Rattus norvegicus
MPMDVILVLWFCVCTARTVLGFGMDPDLQLDIISELDLVNTTLGVTQVAGLHNASKAFLFQDVQREIHSAPHVSEKLIQLFRNKSEFTFLATVQQKPSTSGVILSIRELEHSYFELESSGPREEIRYHYIHGGKPRTEALPYRMADGQWHKVALSVSASHLLLHIDCNRIYERVIDPPETNLPPGSNLWLGQRNQKHGFFKGIIQDGKIIFMPNGFITQCPNLNRTCPTCSDFLSLVQGIMDLQELLAKMTAKLNYAETRLGQLENCHCEKTCQVSGLLYRDQDSWVDGDNCGNCTCKSGAVECRRMSCPPLNCSPDSLPVHISGQCCKVCRPKCIYGGKVLAEGQRILTKTCRECRGGVLVKITEACPPLNCSAKDHILPENQCCRVCPGHNFCAEAPKCGENSECKNWNTKATCECKNGYISVQGNSAYCEDIDECAAKMHYCHANTVCVNLPGLYRCDCVPGYIRVDDFSCTEHDDCGSGQHNCDKNAICTNTVQGHSCTCQPGYVGNGTICKAFCEEGCRYGGTCVAPNKCVCPSGFTGSHCEKDIDECAEGFVECHNYSRCVNLPGWYHCECRSGFHDDGTYSLSGESCIDIDECALRTHTCWNDSACINLAGGFDCLCPSGPSCSGDCPHEGGLKHNGQVWILREDRCSVCSCKDGKIFCRRTACDCQNPNVDLFCCPECDTRVTSQCLDQSGQKLYRSGDNWTHSCQQCRCLEGEADCWPLACPSLGCEYTAMFEGECCPRCVSDPCLAGNIAYDIRKTCLDSFGVSRLSGAVWTMAGSPCTTCKCKNGRVCCSVDLECIENN

SEQ ID NO: 7 - DNA, 18 bp, Artificial
  synthetic PCR primer
tgcagcagaagccgtcca

SEQ ID NO: 8 - DNA, 20 bp, Artificial
  synthetic PCR primer
caaactagggcaagctagag

SEQ ID NO: 9 - DNA, 25 bp, Artificial
  synthetic PCR primer
gcagagacgagacttggtcaactgg

SEQ ID NO: 10 - DNA, 20 bp, Artificial
  synthetic PCR primer
gtgttgtgcttgtggttacc

SEQ ID NO: 11 - DNA, 20 bp, Artificial
  synthetic PCR primer
atagaccaggggcagaaacc

SEQ ID NO: 12 - DNA, 20 bp, Artificial
  synthetic PCR primer
gtgttgtgcttgtggttacc

(19) United States
(12) Patent Application Publication
(10) Pub. No.: US 2006/0053503 A1
(43) Pub. Date: Mar. 9, 2006

(54) Cranial and vertebral defects associated with loss-of-function of Nell

(75) Inventors: Cymbeline T. Culiat, Oak Ridge, TN (US); Eugene M. Rinchik, Holmes, NY (US)

Correspondence Address: QUARLES & BRADY, 411 EAST WISCONSIN AVENUE, MILWAUKEE, WI, 53202-4497, US

(73) Assignee: UT-Battelle, LLC

(21) Appl. No.: 11/192,813

(22) Filed: Jul. 29, 2005

Related U.S. Application Data
(60) Provisional application No. 60/592,552, filed on Jul. 30, 2004.

(51) Int. Cl. A01K 67/027; A61K 38/17; C07K 14/705; C12N 5/06

(52) U.S. Cl. 800/18; 435/354; 530/350; 514/12

(57) ABSTRACT

The mouse Nell1 cDNA and amino acid sequences are disclosed. Also disclosed is a Nell1 knock-out mouse with several bone- and cartilage-related defects. On the molecular level, the loss of Nell1 function led to reduced expression of certain extracellular matrix proteins. The disclosure here provides new tools for studying bone and cartilage development as well as new drug screening and treatment strategies for bone- and cartilage-related diseases and conditions.

### CROSS-REFERENCE TO RELATED APPLICATIONS

This application claims the benefit of U.S. provisional application 60/592,552, filed on Jul. 30, 2004.

### STATEMENT REGARDING GOVERNMENT LICENSE RIGHTS

The U.S. Government has a paid-up license in this invention and the right in limited circumstances to require the patent owner to license others on reasonable terms as provided for by the terms of contract Nos. DE-AC05-000R22725 and KP1104010 awarded by U.S. Department of Energy.

### BACKGROUND OF THE INVENTION

Nell1 is a protein kinase C (PKC) β-binding protein (Kuroda, S. & Tanizawa, K. Biochem. Biophys. Res. Commun. 265, 752-757, 1999, incorporated herein by reference in its entirety). The Nell1 cDNA and amino acid sequences from a variety of mammalian species are available. For example, human Nell1 cDNA can be found at GenBank Accession No. BC096102 (SEQ ID NO:3 and the corresponding amino acid sequence is provided as SEQ ID NO:4) and rat Nell1 cDNA can be found at GenBank Accession No. NM_{—}031069 (SEQ ID NO:5 and the corresponding amino acid sequence is provided as SEQ ID NO:6). The full length mouse Nell1 gene corresponding to the above human and rat sequences, however, has not been identified and cloned.

Overexpression of Nell1 has been shown to cause premature fusion of the growing cranial bone fronts, resulting in craniosynostosis in humans and transgenic mice carrying a rat Nell1 transgene (Zhang, X. et al. J. Clin. Invest. 110, 861-870, 2002; and Ting, K. et al. J. Bone Miner. Res. 14, 80-89, 1999). It is not known, however, what an effect a loss of NELL1 function will have on mammalian animals. In addition, PKC-β has been shown to localize in the vertebrate bodies and intervertebral disc spaces of human fetuses during the 8^{th} week of development, a critical development period when chondrogenetic and osteogenetic processes are initiated in the vertebral column (Bareggi, R. et al. Boll. Soc. Ital. Biol. Sper. 71, 83-90, 1995). It is currently not known whether alteration in Nell1 activity will affect spinal development and structure.

### BRIEF SUMMARY OF THE INVENTION

The mouse Nell1 cDNA and amino acid sequences are disclosed. Also disclosed is a Nell1 knock-out mouse with several bone- and cartilage-related defects. On the molecular level, the loss of Nell1 function led to reduced expression of certain extracellular matrix proteins. The disclosure here provides new tools for studying bone and cartilage development as well as new drug screening and treatment strategies for bone- and cartilage-related diseases and conditions.

### BRIEF DESCRIPTION OF THE SEVERAL VIEWS OF THE DRAWINGS

FIG. 1a shows the phenotype of l7R6^{6R} homozygote mutants at 19 days of gestation. On the right is a fetus homozygous for the l7R6^{6R} allele (from stock 102DSJ) showing a very curled position, enlarged head size and a more spherical head shape, compared to the control littermate (left). l7R6^{6R} mouse fetuses are recovered alive by caesarean rescue because they do not survive delivery through the birth canal perhaps due to the physical trauma in the neck and spine region brought about by the abnormal spinal curvature.

FIG. 1b shows complementation analysis showing the mapping of the l7R6 locus into an interval in mouse chromosome 7 (grey box) that is homologous to a segment of human chromosome 11p15 (grey box) where the Nell1 gene is located. Mouse chromosome 7 is represented by the line with a filled circle at the left (indicating the centromere) and relative positions of genes and markers are indicated above the line. Five mutant mouse lines carrying deletions of varying lengths and surrounding the pink-eyed dilution gene (p) are shown as 46DFiOD, 47DTD, 2MNURf, 8R250M and 3R30M. Among these mutations only the 3R30M deletion can complement the ENU-induced mutations at l7R6 indicating that this deletion does not extend to the position where the l7R6 gene is located. The interval is therefore defined by the proximal deletion breakpoints of the 8R250M and 3R30M mutant mouse lines.

FIG. 2a shows Nell1 expression profiles in heads (H) and bodies (B) of wild-type embryos/fetuses (samples 1-8) and adult mouse tissues (samples 9-16). Samples are as follows: 1, E10; 2, E12; 3, E14H; 4, E14 B; 5, E16H; 6, E16 B; 7, E18H; 8, E18 B; 9, brain; 10, liver; 11, spleen; 12, kidney; 13, thymus; 14, heart; 15, lung; 16, muscle. The Nell1 cDNA probe detects a 3.5-kb transcript as early as E10 days. From E14-E18 days, the Nell1 message is abundant in both fetal heads and bodies, increasing dramatically in the head as development proceeds. Hybridization of the blot with an actin probe serve as control to compare levels of samples loaded in each lane.

FIG. 2b shows Northern blot analysis on polyA+RNAs extracted from the heads of hemizygous E15 l7R6 embryos. A severely reduced expression of the Nell1 gene in the l7R6^{6R} (102DSJ) allele was observed when compared to normal levels of expression detected in mice with the following genotypes: wild-type, mutant hemizygote carrying an ENU-induced mutation in a gene linked to the p region (335SJ), and the three original alleles at the l7R6 locus (88SJ, 435DSJ, 2038SJ).

FIG. 3a shows mouse Nell1 cDNA sequence (part of SEQ ID NO:1), the corresponding amino acid sequences (SEQ ID NO:2), and protein domains. The location of the ENU-induced mutation at bp No. 1546 in the cysteine codon (amino acid No. 502) are both shown. The premature termination codon introduced at this site will truncate the protein and remove the EGF-like domains that are essential for the binding to PKC β1.

FIG. 3b shows sequence electropherograms and the identification of the 102DSJ mutation. The wild-type sequence is shown on the left while the mutant sequence is on the right. Arrows indicate the position of the T to A base change.

FIG. 4a shows skeletal defects in l7R6^{6R}/Nell1^{6R} homozygote mutant mouse (right) at 18 days of gestation. There is alteration of spinal curvature, decreased in intervertebral disc spaces, reduced thoracic volume, protruding sternum and a slight enlargement of the skull.

FIG. 4b is a closeup of the cervical region where the most pronounced vertebral compression is located.

### DETAILED DESCRIPTION OF THE INVENTION

The present invention is based on the inventors' cloning and determination of the full length cDNA sequence of the mouse Nell1 gene and the generation of Nell1 knock-out mice. The inventors observed that, in comparison to normal control mice, the Nell1 knock-out mice had altered cranial morphology, overgrowth of the parietal and frontal calvarial bones, altered spinal curvature, decreased intervertebral spaces, reduced thoracic volume, and raised ribs. The defects in the vertebral column and rib cage of Nell1 knock-out mice indicate that Nell1 plays an important role in endochondral ossification. In addition, the inventors determined that the loss of Nell1 function reduces the expression primarily of genes coding for the extracellular matrix proteins such as specific collagens, tenascins, thrombospondins, and proteoglycan. Without intending to be limited by theory, the inventors believe that the reduced expression of extracellular matrix proteins contributed at least partially to the reduction in intervertebral spaces in the spine. Given that the structure and function of Nell1 is highly conserved among mammalian species, which is supported by the mouse Nell1 sequence provided herein, the phenotype of the Nell1 knock-out mice is believed to be highly relevant and applicable to other mammalian species including humans and rats.

In one aspect, the present invention relates to an isolated nucleic acid that comprises an uninterrupted nucleotide coding sequence that encodes the mouse NELL11 protein as defined by the amino acid sequence of SEQ ID NO:2. Preferably, the nucleotide coding sequence is the mouse Nell1 cDNA (nucleotides 40-2469 of SEQ ID NO: 1). Optionally, the isolated nucleic acid further comprises a transcription control sequence (e.g., a non-native transcription control sequence) such as a promoter operably linked to the coding nucleotide sequence. A host cell comprising the above nucleic acid is also within the scope of the present invention.

In another aspect, the present invention relates to an isolated polypeptide that comprises the amino acid sequence of the mouse NELL1 protein as defined by SEQ ID NO:2. In a related aspect, the present invention relates to an antibody, polyclonal or monoclonal, that specifically binds the mouse NELL1 protein. By specifically binding the mouse NELL1 protein, we mean that the affinity of the antibody for the mouse NELL1 protein is at least one fold, preferably at least five-fold, and most preferably at least 10-fold, higher than that for the NELL1 protein of another mammalian species.

The term “isolated nucleic acid” or “isolated polypeptide” used in the specification and claims means a nucleic acid or polypeptide isolated from its natural environment or prepared using synthetic methods such as those known to one of ordinary skill in the art. Complete purification is not required in either case. Nucleotide or amino acid sequences that flank a nucleic acid or polypeptide in nature can but need not be absent from the isolated form. A nucleic acid and polypeptide of the invention can be isolated and purified from normally associated material in conventional ways such that in the purified preparation the nucleic acid or polypeptide is the predominant species in the preparation. At the very least, the degree of purification is such that the extraneous material in the preparation does not interfere with use of the nucleic acid or polypeptide of the invention in the manner disclosed herein. The nucleic acid or polypeptide is preferably at least about 85% pure, more preferably at least about 95% pure, and most preferably at least about 99% pure.

Further, an isolated nucleic acid has a structure that is not identical to that of any naturally occurring nucleic acid or to that of any fragment of a naturally occurring genomic nucleic acid spanning more than three separate genes. The term therefore covers, for example, (a) a DNA that has the sequence of part of a naturally occurring genomic DNA molecule but which is not flanked by both of the coding sequences that flank that part of the molecule in the genome of the organism in which it naturally occurs; (b) a nucleic acid incorporated into a vector or into the genomic DNA of a prokaryote or eukaryote in a manner such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. Specifically excluded from this definition are nucleic acids present in mixtures of (i) DNA molecules, (ii) transfected cells, and (iii) cell clones, e.g., as these occur in a DNA library such as a cDNA or genomic DNA library. An isolated nucleic acid molecule can be modified or unmodified DNA or RNA, whether fully or partially single-stranded or double-stranded or even triple-stranded. A modified nucleic acid molecule can be chemically or enzymatically induced and can include so-called non-standard bases such as inosine.

In another related aspect, the present invention relates to a genetically engineered mouse cell in which the Nell1 nucleic acid sequence has been disrupted. For the purpose of the present invention, a disrupted Nell1 nucleic acid sequence means that one or more mutations have been introduced into the sequence so that no detectable level of functional NELL1 protein is expressed from the sequence. One or both chromosomal copies of the Nell1 nucleic acid sequence can be disrupted in the cell. In one embodiment, the mouse cell is selected from an osteoblast precursor cell or a chondrocyte precursor cell. The term osteoblast precursor cell is used broadly here to cover any cell that can be induced to differentiate into an osteoblast including, for example, an embryonic stem cell, a mesenchymal stem cell, an osteoprogenitor cell, or a preosteoblast. Similarly, the term chondrocyte precursor cell is used broadly to cover any cell that can be induced to differentiate into a chondrocyte including, for example, an embryonic stem cell, a mesenchymal stem cell, or a chondroprogenitor cell. It is well established in the art that embryonic stem cells and mesenchymal stem cells can be induced to differentiate into osteoblasts and chondrocytes (see e.g., Kale, S. et al. Crit. Rev. Eukaryot. Gene Expr. 10:259-271, 2000; Barberi, T. et al. PLoS Med. 2(6):e161, 2005; Williams, C. G. et al. Tissue Eng. 9:679-88, 2003; Bergman, R. J. J. Bone Miner. Res. 11:268-577, 1996; and Kale s et al. Nat Biotechnol. 18:954-958, 2000). Progenitor cells that can be induced to generate osteoblasts and chondrocytes have also been isolated from the bone marrow (see e.g., Muschler, G. F. et al. J. Orthop. Res. 19:117-25, 2001; D'Ippolito, G. et al. J. Bone Miner. Res. 14:1115-22, 1999; Owen, J. Cell Sci. Suppl. 10:63-76, 1988; and U.S. Pat. No. 5,226,914). In another embodiment, the cell is selected from an osteoblast, an osteocyte, or a chondrocyte.

In one embodiment, the Nell1 knock-out cell does not express any part of the Nell1 coding nucleic acid sequence at the mRNA level.

In another aspect, the present invention relates to a mouse that does not produce a detectable level of functional mouse NELL1 protein (referred to as Nell1 knock-out mouse for the purpose of the present invention) wherein the mouse is characterized by altered spinal curvature, decrease intervertebral space, or both. Such a mouse can be made by, for example, disrupting the Nell1 nucleic acid sequence. The term knock-out mouse is used here broadly to encompass a knock-out fetus (e.g., a E10-E21 fetus, a E15-E21 fetus, a E15-E20 fetus, a E17-E21 fetus, a E17-E20 fetus, a E17-E19 fetus, a E18 fetus, or a E19 fetus) as well as a knock-out neonate. The gestation period for mice is typically between 17 to 21 days.

The mouse Nell1 gene may be disrupted using a variety of technologies familiar to those skilled in the art. For example, a stop codon may be introduced into the gene by homologous recombination. In one embodiment, the stop codon is introduced prior to codon 550 (e.g., at codon 502 described in the example below). Alternatively, a deletion may be introduced into the gene by homologous recombination. In some embodiments, stop codons may be introduced in all reading frames in the sequence downstream of the deletion to eliminate artifactual translation products. In further embodiments, the gene may be disrupted by inserting a gene encoding a marker protein, for example, therein via homologous recombination.

In one embodiment, the knock-out mouse of the present invention does not express any part of the Nell1 coding nucleic acid sequence at the mRNA level.

A skilled artisan is familiar with how a mouse or mouse cell with disrupted Nell1 gene can be generated. For example, the generation of a knock-out mouse can involve the production of a suitable gene-targeting vector, the isolation of correctly genetically modified embryonic stem cells, the provision of mouse blastocysts with these cells by way of injection, the establishment of chimeras and the pairing of these mice to generate mice having the desired genotype (A. L. Joyner: Gene targeting: A practical approach, Oxford University Press, Oxford, 1993, p. 1-234).

In addition to disrupting the Nell1 gene nucleic acid sequence as described above, the Nell1 gene can also be inactivated according to other methods known to a person skilled in the art. The use of the antisense technique or the injection of neutralizing antibodies are examples of such other methods.

Since the Nell1 knock-out mutant is typically expected to be neonatal lethal, it is preferred that a Nell1 knock-out fetus, full term or not (e.g., a E15-E20 fetus, a E17-E19 fetus, a E18 fetus, or a E19 fetus), be rescued by caesarean section.

In still another aspect, the present invention relates to a method for identifying a biomarker for a disease or condition related to abnormal bone or cartilage development. The method involves providing a human subject having the disease or condition and determining whether the subject carries a mutation in Nell1 gene or whether Nell1 expression in the subject is lower than that of a normal control. In one embodiment, the disease or condition is a cranial defect or spinal anomaly. In another embodiment, the disease or condition is the Ehlers Danlos Syndrome (e.g., type VI Ehlers Danlos Syndrome) or a severe cartilage defect. In still another embodiment, the disease or condition is enlargement of head, spherical head shape, alteration of spinal curvature, decreased intervertebral spaces, reduced thoracic volume, and raised ribs.

In yet another aspect, the present invention relates to a method for identifying an agent that can promote the differentiation of an osteoblast or chondrocyte precursor cell to an osteoblast or chondrocyte. The method involves providing an osteoblast or chondrocyte precursor cell in which the Nell1 nucleic acid sequence has been disrupted, treating the cell with a test agent and a set of conditions known to induce the differentiation of a corresponding normal precursor cell in which the Nell1 sequence is not disrupted into an osteoblast or chondrocyte, and determining whether the treated cell is more differentiated than a control cell not treated with the test agent. An example for inducing mesenchymal stem cells in a polymeric carrier to differentiate into bone or cartilage cells is described in U.S. Pat. No. 6,214,369. Other examples can be found in e.g., Kale, S. et al. Crit. Rev. Eukaryot. Gene Expr. 10:259-271, 2000; Barberi, T. et al. PLoS Med. 2(6):e161, 2005; Williams, C. G. et al. Tissue Eng. 9:679-88, 2003; Bergman, R. J. J. Bone Miner. Res. 11:268-577, 1996; Kale s et al. Nat Biotechnol. 18:954-958, 2000; Muschler, G. F. et al. J. Orthop. Res. 19:117-25, 2001; D'Ippolito, G. et al. J. Bone Miner. Res. 14:1115-22, 1999; Owen, J. Cell Sci. Suppl. 10:63-76, 1988; and U.S. Pat. No. 5,226,914. The agents identified by the method is useful for treating a disease or condition related to abnormal bone or cartilage development.

In a related aspect, the present invention relates to another method for identifying an agent as a candidate for treating a disease or condition related to abnormal bone or cartilage development. In this method, a pregnant female mouse carrying a Nell1 knock-out embryo or fetus is exposed to a test agent for a predetermined period of time and the fetus or neonatal mouse is then analyzed to determine whether a defect selected from enlargement of head, spherical head shape, alteration of spinal curvature, decreased intervertebral spaces, reduced thoracic volume, or raised ribs has been at least partially corrected in comparison to a control Nell1 knock-out fetus or neonatal mouse of the same developmental stage whose mother is not exposed to the test agent. The pregnant female mouse employed in the method can be readily made by breeding heterozygous male and female mice carrying one wild-type Nell1 allele and one Nell1 knock-out allele. The pregnant mouse can be exposed to a test agent during any period of gestation. Exposure to the test agent can be made by, for example, including the agent in the mouse diet, intravenous injection, and other suitable means. Since Nell1 knock-out mutants are unlikely to survive the physical trauma of birth, they are rescued by caesarean section in a preferred embodiment.

In another aspect, the present invention relates to a method for repairing damages to an intervertebral disc or articular cartilage in a human or non-human mammalian animal (e.g., rats, mice, domesticated animals such as horses and cows, and pets such as dogs and cats). Intervertebral discs and articular cartilage can be damaged by injury or lifetime of use. In the case of intervertebral disc herniation, a herniated disc can press on spinal nerves, often also resulting in inflammation. Depending on the location of the disc that is herniated, this can cause pain, numbness, tingling or weakness in the neck, shoulders, arms, back, legs or feet. Severe disc herniation typically requires surgery. However, 70% of the patients who have undergone surgery still suffer from pain and approximately 10% of the patients have to repeat the surgery over the years. Intervertebral discs also tend to degenerate over time and that is why old people “grow shorter.” In the case of articular cartilage damage, it does not heal as rapidly or effectively as other tissues in the body. Instead, the damage tends to spread, allowing the bones to rub directly against each other and resulting in pain and reduced mobility. The treatment provided here for damages to an intervertebral disc or articular cartilage involves administering NELL1 protein or chondrocytes genetically engineered to overexpress NELL1 protein to an intervertebral disc or a joint.

When NELL1 protein is administered to a human or non-human animal, it can be injected directly to an intervertebral disc or joint including an area adjacent to the disc or joint cartilage. In this regard, NELL1 protein can be injected into, for example, the epidural space utilizing a spinal needle. NELL1 protein can also be administered indirectly to an intervertebral disc or joint through an another route such as intravenous injection.

NELL1 protein can be administered in an extended-release formulation. Suitable extended release formulations may comprise microencapsulation, semi-permeable matrices of solid hydrophobic polymers, biodegradable polymers, and biodegradable hydrogels, suspensions or emulsions (e.g., oil-in-water or water-in-oil). Optionally, the extended-release formulation comprises poly-lactic-co-glycolic acid (PLGA) and can be prepared as described in Lewis, “Controlled Release of Bioactive Agents form Lactide/Glycolide polymer,” in Biodegradable Polymers as Drug Delivery Systems, M. Chasin & R. Langeer, Ed. (Marcel Dekker, New York), pp. 1-41. Optionally, a stabilizing agent such as a water-soluble polyvalent metal salt can be included in the extended release formulation. Many examples of the extended-release formulations are described in U.S. Pat. No. 6,689,747, which is herein incorporated by reference in its entirety.

Any chondrocytes that are genetically engineered to overexpress a NELL1 protein can be used in the present invention for transplantation to an intervertebral disc or articular joint. The chondrocytes can be those isolated from a cartilage or those obtained by inducing the differentiation of chondrocyte precursor cells such as embryonic stem cells or mesenchymal stem cells. Both of these methods are mature technology in the art (see e.g., Ganey, T. et al. Spine 28:2609-2620, 2003; Williams, C. G. et al. Tissue Eng. 9:679-88, 2003; Bergman, R. J. J. Bone Miner. Res. 11:268-577, 1996; Kale, S. et al. Nat. Biotechnol. 18:954-958, 2000; Barberi, T. et al. PLoS Med. 2(6):e161, 2005; Owen, J. Cell Sci. Suppl. 10:63-76, 1988; U.S. Pat. No. 6,214,369; and U.S. Pat. No. 5,226,914). To make chondrocytes that overexpress a NELL1 protein, an expression vector carrying a NELL1 encoding nucleic acid (preferably the NELL1 of the same species) can be introduced into the chondorcytes. Alternatively, a genetic construct for overexpressing NELL1 (preferably the NELL1 of the same species) can be integrated into the genome of the chondrocytes. It is mature technology to transplant chondrocytes to intervertebral discs or articular joints (see e.g., U.S. 2002/0091396). In this regard, chondrocytes can be provided as an cartilage implant (see e.g., U.S. Pat. No. 6,852,331 and U.S. Pat. No. 5,928,945). To minimize the problem of tissue rejection, it is preferred that the autologous chondrocytes are transplanted. Autologous disc chondrocytes removed from damaged cartilage tissue remain a capacity to proliferate, produce, and secrete matrix components (Ganey, T. et al. Spine 28:2609-2620, 2003). Typically, chondrocytes can be removed from a cartilage, genetically engineered to overexpress NELL1, expanded in culture, and transplanted back to repair disc damage or disc degeneration.

The invention will be more fully understood upon consideration of the following non-limiting example.

### EXAMPLE

### Loss of Function in the Mouse Nell1 Gene Reduces Expression of Extracellular Matrix Proteins Resulting in Cranial and Vertebral Defects

This example describes the generation, position cloning and characterization of Nell1^{6R}, a new, recessive neonatal-lethal point mutation in the mouse Nell1 gene, induced by N-ethyl-N-nitrosourea (ENU). Nell^{6R} has T→A base change that converts a codon for cysteine into a premature stop codon [Cys(502)Ter], resulting in severe truncation of the predicted protein product and marked reduction in steady state levels of the transcript, most likely due to nonsense-mediated decay. In addition to alterations of cranial morphology, Nell^{6R} mutants also manifest skeletal defects in the vertebral column and ribcage, revealing a role for Nell1 in signal transduction in endochondral ossification. Quantitative real-time PCR assays of 219 genes revealed an association between the loss of Nell1 function and reduced expression of genes for extracellular matrix proteins, several of which are involved in the human cartilage disorder Ehlers-Danlos Syndrome.

Materials and Methods

Mouse Breeding and Maintenance: All animals were bred at the Mammalian Genetics Research Facility at Oak Ridge National Laboratory (ORNL), Oak Ridge, Tenn., using protocols approved under the ORNL Institutional Animal Care and Use Committee. The identification and fine-structure mapping of the l7R6 locus in mouse Chr 7 are described in Rinchik, E. M. et al. Proc. Natl. Acad. Sci. 99:844-849, 2002. The 88SJ (l7R6^{1R}), 335SJ (l7R6^{2R}), 2038SJ (l7R6^{3R}) mutations (m) were induced on ru2 p chromosomes from the non-inbred, closed-colony stock BJR, while the 102DSJ allele (l7R6^{6R}), was induced in the p chromosome from the non-inbred, closed-colony 21A strain. To generate the mutant hemizygotes from the SJ lines, progeny-tested males carrying the ENU-induced mutation (Hps5^{ru2}++/Hps5^{ru2} m p) were mated to ++p^{7R}/Hps5^{ru2} Del(Hps5^{ru2} p)^{46DFiOD} females. For 102DSJ, progeny-tested +p^{7R}/l7R6^{6R} p males were mated with +p^{7R}/Del(Hps5^{ru2} p)^{46DFiOD}. Matings were done for one hour early in the morning, and females were examined for the presence of vaginal plugs (gestation day 0). Embryos were collected at 15, 18, and 19 days of gestation. Females of these strains usually deliver at 19 days of gestation, so neonates (P0) were also collected along with E19 fetuses recovered by caesarean section. Mutant hemizygotes [Hps5^{ru2} m p/Del(Hps5^{ru2}p)^{46DFiOD} or l7R6^{6R} p/Del(Hps5^{ru2}p)^{46DFiOD}] are distinguishable from wild-type and heterozygous littermates by three criteria: the non-pigmented eye coloration and by molecular genotyping with for size polymorphisms using D7Mit70 and D7Mit315, microsatellites tightly linked to the p gene. The 102DSJ mutation was recovered in a manner similar to that described previously for the 88SJ, 335SJ, and 2038SJ alleles at the l7R6 locus (Rinchik, E. M. et al. Proc. Natl. Acad. Sci. 99:844-849, 2002) Mutagenized chromosomes marked with the p mutation were recovered in G1 females from ENU-treated 21A G0 males. The 102DSJ lethal mutation was recognized when G1 female #102 failed to yield any pink-eyed-dilute G2 progeny when she was crossed to a +p^{7R}/Del(Hps5^{ru2} p)^{46DFiOD} G1 male. Deletion mapping also similar to that performed previously (Rinchik, E. M. et al. Proc. Natl. Acad. Sci. 99:844-849, 2002) revealed that the 102DSJ lethal mapped to the same deletion interval as did the previously ascertained l7R6 alleles. Allelism was confirmed (i.e., 102DSJ=l7R6^{6R}) when no pink-eyed dilute progeny were found in >30 progeny of a cross of 88SJ (Hps5^{ru2} l7R6^{1R}p/Hps5^{ru2}++) and 102DSJ (+102DSJ p/++p^{7R}) heterozygotes, when 25% were expected (p<0.001).

Skeletal Staining: Skeletal defects were evaluated using the alizarin red-alcian blue staining protocol (Hogan, B., Beddington, R., Constantini, F. & Lacy, E. 379-380, Cold Spring Harbor Press, New York, 1994). Embryos were briefly soaked in 70° C. water and the skin and internal organs were removed. Embryos were fixed in 95% ethanol, stained in Alcian Blue for 1-2 days and rinsed in 95% ethanol. They were then cleared in 1% KOH (2-6 hrs), subsequently stained for 3 h in alizarin red solution, and cleared further by placing in 2% KOH overnight. Clearing was completed by processing through the following series of solutions of 2% KOH/glycerol: (80:20), (60:40), (40:60), and (20:80) with storage indefinitely in the final solution.

Histology: Haematoxylin and Eosin staining. Luxol Fast Blue-Periodic Acid Schiff Stain (LFB-PAS) and Masson Staining of sections of E19 embryos from mutant and wild-type were conducted according to standard histological protocols.

RNA Analysis: Total RNAs were extracted from fetuses and adult tissues using standard guanidine isothiocyanate procedures (Ausubel, F. M., Brent, R., Kingston, R. E., Moore, D. D. & G., S. J. Current Protocols in Molecular Biology, John Wiley & Sons, New York). Phase Lock Gels™ (Eppendorf) were used for subsequent phenol-chloroform purifications. RNA was precipitated with isopropanol and after centrifugation pellets were re-suspended in nuclease-free water. About 700 μg-1 mg total RNA per sample was used for purifying polyA⁺ RNA using Mini-Oligo(dt) Cellulose spin columns (5 Prime-3 Prime, Inc.). One-2 μg of polyA⁺ RNAs were used for Northern Blots using standard electrophoresis and blotting protocols (Sambrook, J., Fritsch, E. F. & Maniatis, T. Molecular Cloning: A Laboratory Manual, Cold Spring Harbor Laboratory Press, 1989). Blots were hybridized with the CTC55+59 probe, which was generated by RTPCR using primers designed based on mouse EST sequences matching the 5′ and 3′ ends of human NELL1 (1920 bp; ctc 55-TGCAGCAGAAGCCGTCCA (SEQ ID NO:7); ctc 59 CAAACTAGGGCAAGCTAGAG (SEQ ID NO:8)).

DNA Analysis and Sequencing: Templates for sequencing were either cloned or PCR-amplified cDNA segments. First strand cDNA templates were generated from poly A+RNAs extracted from E15 fetal heads using the RETROscript Kit (Ambion). Overlapping cDNAs segments covering the entire coding region plus the 5′ and 3′-untranslated region were generated using the following primer pairs: ctc 55+59 (1920 bp; ctc

(SEQ ID NO:7)

| | |
|---|---|
| 55-TGCAGCAGAAGCCGTCCA; | ctc 59 |

(SEQ ID NO:8)

| | |
|---|---|
| CAAACTAGGGCAAGCTAGAG, | ctc 150 + 151 (ctc 150- |

(SEQ ID NO:9)

| | |
|---|---|
| GCAGAGACGAGACTTGGTCAACTGG; | ctc 151- |

(SEQ ID NO:10)

GTGTTTGTGCTTGTGGTTACC).

Mutation Scanning: Twenty primer sets were designed to amplify each exon of Nell1 from flanking intron sequences and two primers sets for conserved upstream elements. Each amplicon was amplified from genomic DNAs of Nell^{3R} and Nell^{6R} mutant mice, and the control strains, BJR and 21A, respectively. Corresponding PCR products were mixed in equal volumes, heteroduplexed and scanned for point mutations using TGCE (Li, Q. et al. Electrophoresis 23:1499-511, 2002). Three overlapping temperature gradients were used: 50-60° C., 55-62° C., and 60-68° C. The 421 bp amplicon containing the mutation in the l7R6^{6R} allele was amplified by PCR using the following primer pairs designed from the intron sequences flanking the 131 bp exon 14 of Nell1; NellExon14(F): ATAGACCAGGGGCAGAAACC (SEQ ID NO:11) and NellExon14R: TTGCCT CAACCT CAATAT CC (SEQ ID NO:12).

High-Throughput Quantitative real-time PCR assays: RNAs from four E18 102DSJ mutant hemizygotes and four hemizygous wild-type embryos were extracted according to the RNA extraction method described earlier.

RNA Purification and cDNA Synthesis (Isolation method and DNAse treatment): DNAse-treated RNA was ethanol precipitated and resuspended in nuclease-free water. Total RNA (2.5 μg) was converted to cDNA using the random-priming High-Capacity cDNA Archive Kit (Applied Biosystems).

Multiplex Preamplification of cDNA Targets: To enable maximum sensitivity and detection of hundreds of gene expression targets from a small amount of cDNA, a novel multiplex PCR preamplification strategy was used prior to conventional quantitative PCR. 226 (220 experimental and 6 endogenous control) Taqman Gene Expression Assays (PCR primer/FAM-probe stock solutions) were pooled together and used in a single PCR to amplify all targets equally from the same cDNA template. The FAM-probe is a component of the final configuration of the manufactured TaqMan Gene Expression Assays and does not interfere with the preamplification process. To prepare the multiplex preamplification primer pool, equal volumes of the 226 TaqMan® Gene Expression Assays were mixed together, dried under vacuum, and re-suspended with water to generate a multiplex-pooled primer set with a concentration of 180 nM for each primer. The preamplification reaction was set up as follows: A 250 μl volume of 500 ng of cDNA was combined with 250 μl of the multiplex-pooled primers. Then, 500 μl of 2× Multiplex Preamplification Master Mix was added to generate the final 1000 μl reaction volume (Applied Biosystems). The reaction mix was divided into 50 μl aliquots in a 96-well PCR tray and cycled on an ABI 9700 thermalcycler under the following conditions: 95° C. for 10 minutes; then 10 cycles of 95° C. for 15 seconds; and 60° C. anneal/extension for 4 minutes.

Real-Time PCR Reactions: Preamplification products were recombined into one tube and diluted 1:5 with water. Individual singleplex TaqManGene Expression Assays for each of the 226 preamplified markers were prepared as follows: 5.0 μl of 2× TaqMan® Universal PCR Master Mix, 0.5 μl of TaqMan® Gene Expression Assay 20× primer/FAM-probe solution and 2.0 μl of water, and 2.5 μl of preamplified cDNA product. For all samples, each assay was carried out in quadruplicate wells of 384-well plates and run in the ABI PRISM®7900HT Sequence Detection System under two-temperature cycling: 95° C. for 10 minutes, then 40 cycles of 95° C. for 15 seconds and 60° C. for 1 minute. C_{T} (threshold cycle) values, the cycle number at which the PCR amplification fluorescence signal crosses a fluorescence threshold, were generated using the FAM dye layer setting at a threshold of 0.2 and a baseline of 3-13.

Data analysis: The relative levels of transcripts for each gene in wild-type and mutant samples were compared following normalization to endogenous control targets. GeNORM software (Vandesompele et al, 2002) was used to select the two targets with the least variation across samples from a collection of 6 potential endogenous controls (Hprt, Tfrc, Thp, Gus, and Pgk1). Gus and Hprt were selected for heads, while Gus and Pgk1 were selected for bodies. The geometric mean of the selected targets was then used as the reference for determining ΔC_{T} values. For each sample, ΔC_{T} values were determined by the following equation: ΔC_{T Marker}=C_{T Marker}−C_{T Reference}. Statistically significant differences between ΔCT values of wild-type and mutant groups were determined by a two-tailed t test without assuming equal variances and with a P value cutoff of 0.005. ΔΔ C_{T}S were also calculated between wild-type and mutant groups based upon average ΔC_{T} values for each group, and relative fold differences between them were determined by 2^{ˆ-ΔΔC}_{T}[25].

Results

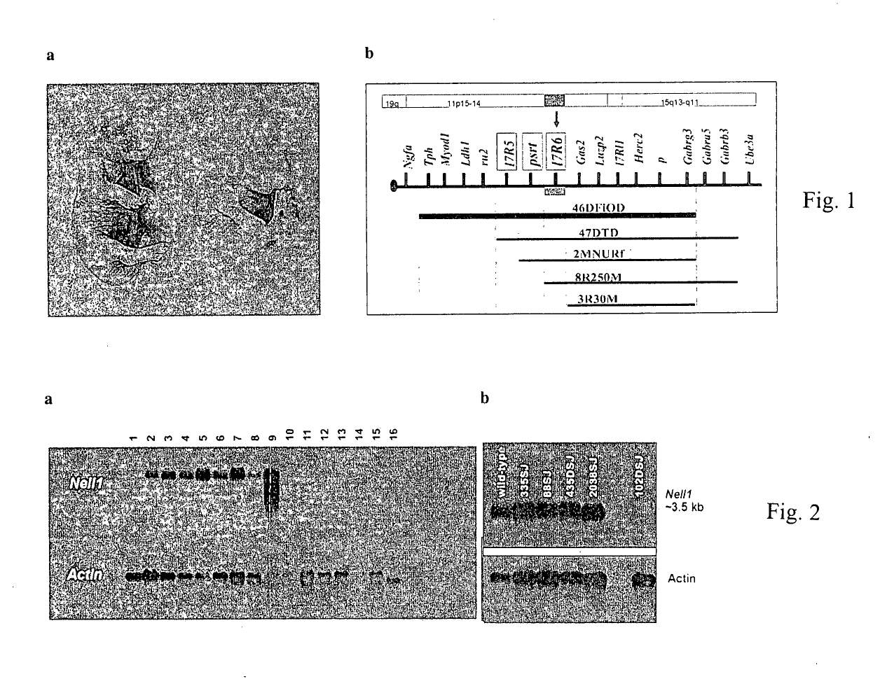

We generated mutant mice with N-ethyl-N-nitrososurea, mapped various lethal mutations to a small segment of mouse chromosome 7, and defined mutations in the l7R6 locus as late gestation/neonatal lethal (Rinchik, E. M. et al. Proc. Natl. Acad. Sci. 99:844-849, 2002). For one allele that we recovered and mapped at this locus, designated l7R66R, the mutants could develop to E19 but were unable to survive the physical trauma of birth. Mutant neonates rescued by caesarean section survived, but quickly succumbed because they are unable to breathe and their foster mothers usually cannibalized them. Late-gestation mutant hemi- or homozygous fetuses and neonates are easily distinguished from normal littermates by a pronounced curled position, enlargement of the head region (FIG. 1a), inability to open their mouths, and very weak reflexes in extremities when stimulated by touching. Heterozygotes survive to adulthood and breed normally, with no readily visible phenotypic differences between l7R6^{6R} heterozygotes and wild-type mice.

Trans complementation analysis with a number of p deletions localized l7R6^{6R} to the same <1 cM segment homologous to a region of human 11p15 (FIG. 1b, Materials and Methods) where several other l7R6 alleles have been mapped. Gene content analysis of this region suggested six candidate genes. One of these genes, NELL1 (NEL-like1 protein expressed in neural tissue encoding an EGF-like domain) was particularly important because it is overexpressed in the prematurely fused sutures of patients manifesting unilateral coronal synostosis. The Nell1 gene encodes a polypeptide (810 amino acids) that is glycosylated and processed in the cytoplasm and then secreted as a 400 kDa trimer. The protein contains several recognizable domains (thrombospondin-like, laminin G, von Willebrand factor-like repeats and epidermal growth factor like (EGF-like)). The NELL1 protein binds to and is phosphorylated by PKC-1, an interaction mediated by the EGF-like domains. This observation suggests that Nell1 represents a new class of ligand molecules critical for growth and development.

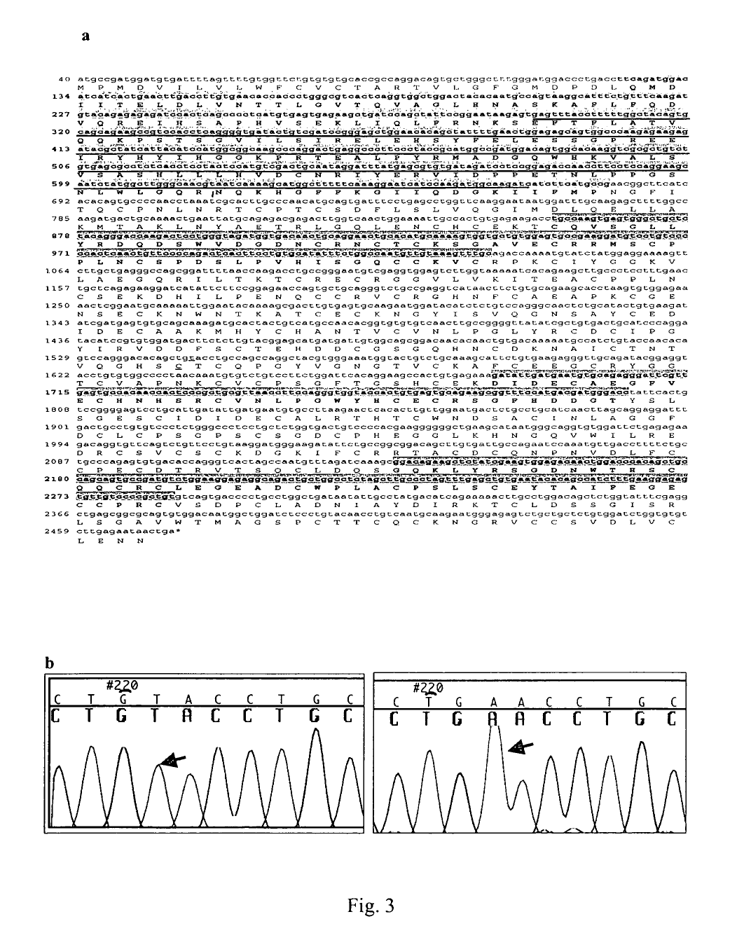

The pronounced enlarged head phenotype, along with the deletion-map position, suggested that recessive l7R6^{6R} mutants may be a loss-of-function allele in the Nell1 gene. Nell1 gene expression was assayed by Northern Blot analysis. The cDNA probe detects a 3.5 kb message in polyA⁺RNA extracted from wild-type embryos from E10-18 days of gestation (FIG. 2a). During gestation, expression steadily increases in the head region and decreases in the body while in adult tissues, expression was observed primarily in adult brain (FIG. 2a). Northern blot assays of RNA samples isolated from E15 fetuses showed barely detectable expression of Nell1 in l7R6^{6R} hemizygotes (FIG. 2b). To identify the presumed Nell1^{6R} (l7R6^{6R}) mutation, each exon along with flanking intron sequences was amplified from genomic DNA and analyzed for single base-pair changes by heteroduplex analysis using temperature gradient capillary electrophoresis (Li, Q. et al. Electrophoresis 23:1499-511, 2002). The presence of heteroduplexes were detected in exon 14 hence the sample was sequenced in mutant animals and compared to the sequence in the wild-type controls (St21a and BJR) (FIG. 3). Sequencing analysis showed a single base pair substitution of T→A that converts a codon for cysteine into a premature stop codon [TGT→TGA; Cys(502)Ter] hence truncating the 810 amino acid protein product. Since transcripts bearing premature stop codons in positions such as the one present in the 102DSJ Nell1 transcript are subject to nonsense mediated decay (Hillman, R. T. et al. Genome Biol. 5:R8, 2004; and Nagy, E. & Maquat, L. E. Trends Biochem. Sci. 23:198-9, 1998), this mutation scanning data is consistent with the severe decrease of RNA levels observed earlier (FIG. 2b).

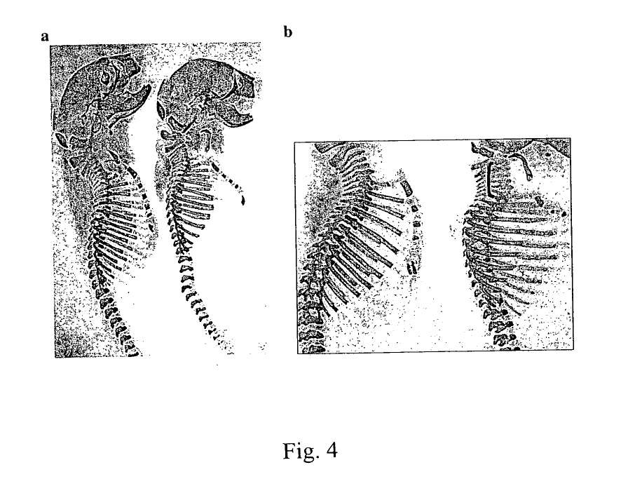

Due to the prior reports on the role of Nell1 in cranial development and osteoblast differentiation we then focused on identifying skull and skeletal defects in the Nell^{6R} mutants by performing morphometric measurements and skeletal analysis using alizarin red-alcian blue staining on E18.5 fetuses recovered by caesarean. Without exception, when compared to their non-mutant littermates, all hemizygous and homozygote mutant fetuses manifest a decrease in body length (crown to rump) due to the pronounced altered curvature of the spine and an enlarged, spherically shaped head brought about by an increase in the head height. Skeletal analysis showed compression of intervertebral spaces and alteration of spinal curvature, shape and volume of the ribcage (FIG. 4). The cervical region of the vertebra displayed the most dramatic reduction in the intervertebral disc material. The profound impact in the development of the vertebral and thoracic skeleton was not anticipated since the deleterious effects of overexpression was confined to the growth and differentiation of the calvarial bones.

In order to define the genes and pathways that are perturbed by the Nell^{6R} mutation high-throughput real time quantitative PCR analysis of 226 genes (219 experimental and 7 controls) were directly assayed in RNA samples extracted from four individual heads and bodies of four E18 102DSJ mutants and four wild-type animals. The genes were carefully selected on the basis of the observed Nell^{6R} phenotype, the putative domains and functions of the Nell1 gene. Moreover, genes associated with craniosynostosis in man and mouse models, skeletal development (bone and cartilage), cell growth and differentiation, neural development and signal transduction pathways were included, if the assays were available.

The gene expression analyses revealed that 13 genes in the head and 28 genes in the body have reduced expression due to the loss of Nell1 gene function. The expression of the following nine genes are affected in both the heads and bodies: collagen 5 alpha 3 subunit (col5a3), tenascin (tnxb), procollagen type XV alpha 1 (col15a1), procollagen type V alpha (col5a1), thrombospondin (thbs3), matrilin 2 (Matn2), tumor necrosis factor factor ligand (Tnfrsf11b), ostoeblast specific factor (Osf2-pending), chondroadherin (Chad). Further analysis of the genes using publicly available tools such DAVID (Database for Annotation, Visualization and Integrated Discovery), gene cards, UCSC genome browser and extensive PUBMED literature searches showed that majority of the genes that have reduced expression due to the Nell1 mutation, code for extracellular matrix (ECM) proteins such as specific collagens, thrombospondins, tenascins and matrilins, etc. These proteins function in providing cell adhesion, communication, imparting strength and flexibility to tissues. In the head, the most severely affected genes are tenascin b (Tnxb) and procollagen type V alpha 3 subunit (Col5a3), which have 2-3 fold reduced expression. Since only eight out of 21 collagens assayed showed significant changes in expression indicates that the loss of Nell1 influences only a specific set of collagen subunits. Another striking result is that mutations in three of the affected genes Tnxb, Col5a1 and Col6a1 the corresponding genes in humans generate Ehlers-Danlos Syndrome (EDS), a severe cartilage defect that occurs as high as 1/5000 individuals and is characterized by hyper-extensibility of the skin and extreme flexibility of joints. EDS patients do not have the ability to make certain components of the connective tissue, particularly fibrillar collagens. There are six distinct EDS clinical syndromes and EDS type VI is distinguished from the rest by having abnormal curvature of the spine (kyphoscoliosis), hypotonia, joint laxity and ocular fragility (Mao, J. R. & Bristow, J., J. Clin. Invest. 107:1063-9, 2001).

The gene expression profile of the Nell^{6R} mutation, defined by qRTPCR assays, is further supported by detailed histological analysis using haematoxylin and eosin, Periodic Acid Schiff (PAS) and Masson staining. Histological analysis showed that in the mutant bone and cartilage development is delayed compared to the wild-type animals. The production of extracellular material surrounding cells in the developing vertebral bone and interverterbal discs is considerably less in the Nell^{6R} mutant mice compared to the wild-type controls.

Along with the over-expression studies, the Nell1 loss-of-function allele described herein demonstrates the involvement of Nell1 in suture development and closure. The developing suture contains undifferentiated proliferating osteogenic stem cells, a proportion of which are recruited to differentiate into osteoblasts at the edges of the calvarial bones. Unmineralized bone matrix is also deposited at these edges. Mature osteoblasts secrete a collagen-proteoglycan matrix that binds calcium salts, which upon mineralization generates new bone from the osteoid matrix. A delicate balance between stem-cell proliferation and differentiation into bone is required so the stem-cell population is maintained until skull growth is complete. Signals from the dura mater directly underneath the skull maintain sutural patency by regulating cell proliferation and collagen production. Two distinct processes appear to be involved in premature suture closure: a) excessive growth of the calvarial bones so two opposing bone growing fronts become very close/overlap; and b) bony fusion of the overlapping bone fronts.

The alteration of spinal curvature and reduction of intervertebral disc spaces in the mutants described herein indicate a role of Nell1 in signal transduction in the developing spine. This conclusion is consistent with the fact that PKC-β1 isozyme localizes in the vertebral bodies and intervertebral disc spaces of human fetuses during the 8^{th} week of development, a critical developmental period when chondrogenetic and osteogenetic processes are initiated in the vertebral column (Bareggi, R. et al. J. Biol. Res. 121:83-90, 1995). PKC activity has also been observed in the fetal mouse vertebral column and is abundant in the more mature cells close to the ossification center and the intervertebral disc spaces. Overexpression of the Nell1 does not appear to disrupt this process but clearly a reduction/absence or malfunctioning of the protein does. Our data also demonstrate that, in addition to its role in intramembranous bone differentiation, Nell1 has a critical function in endochondral ossification in the spine. The conservation of structure and function of Nell1 gene itself suggests that the spinal phenotype could conceivably also be a consequence of human NELL1 loss-of-function mutations, hence, we suggest that linkage studies and mutation scanning in families segregating both cranial defects and spinal anomalies should certainly focus on the Nell1 gene in chromosome 11p15.

The present invention is not intended to be limited to the foregoing example, but encompasses all such modifications and variations as come within the scope of the appended claims.

We claim:

1. An isolated polypeptide comprising an amino acid sequence defined by SEQ ID NO:2.

2. The isolated polypeptide of claim 1, wherein the polypeptide consists of an amino acid sequence defined by SEQ ID NO:2.

3. An antibody that specifically binds the polypeptide of claim 2.

4. An isolated nucleic acid comprising an uninterrupted nucleotide coding sequence or its complement wherein the uninterrupted coding sequence encodes the polypeptide of claim 2.

5. The isolated nucleic acid of claim 4, wherein the uninterrupted nucleotide coding sequence is nucleotides 40 to 2469 of SEQ ID NO:1.

6. The isolated nucleic acid of claim 4 further comprising a transcriptional control sequence operably linked to the uninterrupted coding sequence that encodes the amino acid sequence defined by SEQ ID NO:2.

7. A host cell comprising the nucleic acid of claim 6.

8. A mouse cell in which the mouse Nell1 nucleic acid sequence has been disrupted.

9. The mouse cell of claim 8, wherein the cell is selected from an osteoblast precursor cell or a chondrocyte precursor cell.

10. The mouse cell of claim 8, wherein the cell is selected from an osteoblast, an osteocyte, or a chondrocyte.

11. The mouse cell of claim 8, wherein both alleles of Nell1 are disrupted.

12. A mouse that does not express a detectable level of functional Nell1 protein and characterized by abnormal spine curvature, decrease intervertebral space, or both.

13. The mouse of claim 12, wherein the mouse Nell1 nucleic acid sequence has been disrupted.

14. The mouse of claim 13, wherein the mouse lacks mRNA made from the Nell1 gene sequence.

15. The mouse of claim 13, wherein the Nell1 gene carries a mutation so that a premature stop codon is introduced before codon 550.

16. The mouse of claim 13, wherein the mouse is an E15 to E20 fetus.

17. A method for identifying a candidate biomarker for a disease or condition related to abnormal bone or cartilage development, the method comprising the steps of:
providing a human subject having the disease or condition; and
determining whether the subject carries a mutation in Nell1 gene or whether Nell1 expression in the subject is lower than that of a normal control.

18. The method of claim 17, wherein the disease or condition is a cranial defect or spinal anomaly.

19. The method of 17, wherein the disease or condition is a spinal anomaly.

20. The method of claim 17, wherein the disease or condition is selected from enlargement of head, spherical head shape, alteration of spinal curvature, decreased intervertebral spaces, reduced thoracic volume, raised ribs, or Ehlers Danlos Syndrome.

21. A method for identifying an agent that can promote the differentiation of an osteoblast or chondrocyte precursor cell to an osteoblast or chondrocyte, the method comprising the steps of:
providing an osteoblast or chondrocyte precursor cell according to claim 9;
treating the cell with a test agent and a set of conditions known to induce the differentiation of a corresponding normal precursor cell in which the Nell1 sequence is not disrupted into an osteoblast or chondrocyte; and
determining whether the treated cell is more differentiated than a control cell not treated with the test agent.

22. A method for identifying an agent as a candidate for treating a disease or condition related to abnormal bone or cartilage development, the method comprising the steps of:
providing a pregnant female mouse carrying a Nell1 knock-out embryo or fetus of claim 12;
exposing the pregnant female mouse to a test agent; and
determining whether the fetus' or neonatal mouse's defect selected from enlargement of head, spherical head shape, alteration of spinal curvature, decreased intervertebral spaces, reduced thoracic volume, or raised ribs has been at least partially corrected in comparison to a control Nell1 knock-out fetus or neonatal mouse of the same developmental stage whose mother is not exposed to the test agent.

23. A method for treating damages to an intervertebral disc or articular cartilage in a human or non-human animal, the method comprising the step of:
administering NELL1 protein or chondrocytes genetically engineered to overexpress NELL1 protein to an intervertebral disc or a joint.

24. The method of claim 23, wherein the chondrocytes are autologous cells.

25. The method of claim 23, wherein the method is for treating a human.
